(19) United States
(12) Patent Application Publication
(10) Pub. No.: US 2009/0035758 A1
(43) Pub. Date: Feb. 5, 2009

(54) Method of Measurement of Micronucleated Erythrocyte Populations

(75) Inventors: Joseph Wolfers, La Bouilladisse (FR); Andreas Van Agthoven, Marseille (FR); Fabrice Malergue, Marseille (FR); Michel Herbert, Marseille (FR)

Correspondence Address: BECKMAN COULTER, INC.;Mitchell E. Alter, P.O. BOX 169015, MAIL CODE 32-A02, MIAMI, FL, 33116-9015, US

(73) Assignee: BECKMAN COULTER, INC., Fullerton, CA (US)

(21) Appl. No.: 11/832,719

(22) Filed: Aug. 2, 2007

(51) Int. Cl. C12Q 1/68

(52) U.S. Cl. 435/6

(57) ABSTRACT

A method of measuring micronucleated erythrocyte populations is disclosed. The method includes contacting a sample containing erythrocyte populations with a fluorescently labeled antibody specific to immature erythrocytes among erythrocyte populations; adding an aqueous permeation reagent to the sample and incubating the formed permeation sample mixture for an incubation time sufficient to allow the permeation reagent to render cellular membrane of the erythrocyte populations permeable to RNase and dye; adding a RNase reagent to degrade RNA and to inhibit further reaction of permeation reagent; adding a fluorescent nucleic acid dye reagent to stain DNA representing micronuclei in the erythrocyte populations; performing light scatter and fluorescence measurements of the final sample mixture on a flow cytometer; differentiating micronucleated erythrocyte populations from other cell types; and reporting the micronucleated erythrocyte populations of the sample. The method avoids ultracold fixation, centrifugation, and washing cells in sample preparation.

### FIELD OF THE INVENTION

The present invention relates to a method of measuring erythrocyte populations, particularly micronucleated erythrocyte populations.

### BACKGROUND OF THE INVENTION

Following exposure of a dividing eukaryotic cell to a genotoxic compound (a compound that causes damage to genomic DNA), various events may occur that damage the integrity of the genome. More specifically, if the compound is a clastogen, chromosome(s) will suffer from DNA double-strand breakage. If the compound is an aneugen, which interferes with the mitotic apparatus, an entire chromosome may lag behind during mitosis or meiosis. In both instances, the end result will be the appearance in the cytoplasm of the daughter cell of either fragment(s) or an entire chromosome that has not segregated correctly into the nucleus of the dividing cells. This fragment(s)/chromosome will then form what is known to be a micronucleus, i.e., a small nucleus which will coexist with the normal nucleus of the cell.

Therefore, during the drug discovery process, or more widely, during development of chemical compounds, analysis of DNA damage is an essential read-out for genotoxicology safety studies.

Mammalian erythrocytes have the particularity of being devoid of nucleus (enucleated). In the bone marrow, during erythropoeisis when an erythroblast matures into a reticulocyte, it expels its nucleus, while retaining other intracellular organelles and cytoplasmic RNA. However, a micronucleus, if present, is not submitted to this expulsion, and will remain in the reticulocyte. Reticulocytes then exit the bone marrow into the blood stream where they represent less than 5% of all erythrocytes, and mature into erythrocytes, gradually loosing their RNA content, while still retaining the micronucleus. Therefore, blood sample analysis of animals having received suspected genotoxic compounds, gives information regarding the genotoxicity of this compound.

Interestingly, some mammals, namely rats and humans, have the characteristic of eliminating micronucleated erythrocytes in the spleen. This adds a level of complexity, in that accurate identification of micronucleated erythrocytes should be limited to the youngest fraction of erythrocytes, i.e., reticulocytes that have not yet been selectively eliminated from the blood stream.

Microscopic analysis of blood samples using a nucleic acid stain such as acridine orange or Pyronin Y is the classical method for identification of micronucleated red blood cells. However, a major problem to this method is the paucity of micronucleated cells among erythrocytes; indeed, their frequency can vary from 2 to 5% in treated rodents to less than 0.5% in normal rodents. If one adds to this the need to specifically analyze only the reticulocyte fraction of erythrocytes, one can see that micronucleated reticulocytes can represent at the most 0.02% of all erythrocytes. This is a major obstacle to high throughput requirement, as microscopic enumeration is extremely time-consuming, and requires expertise. Furthermore, manual counting does not enable to count a statistically significant amount of micronucleated erythrocytes for optimal data analysis.

Recently, part of this drawback has been addressed by laser scanning microscopic techniques, as proposed by Cellomics. However, these techniques require sophisticated hardware, and expertise in data analysis.

Flow cytometry allows multiparametric detection of surface and intracellular molecules at the single cell level. Furthermore, standard flow cytometers can analyze up to 3000 events per second, which drastically reduces the time of analysis, while at the same time increases precision due to the high number of events analyzed.

U.S. Pat. No. 5,229,265 (to Tometsko, et al.) discloses a flow cytometric method of analyzing micronucleated erythrocytes in blood or bone marrow samples. Tometsko, et al. teach to fix the blood or bone marrow cells with an organic fixative at ultra low temperatures of less than −30° C. to cause the cells to exhibit both permeability to fluorescent dyes and compatibility with flow cytometry analysis. The method utilizes two nucleic acid dyes to stain the fixed cells, wherein one dye selectively stains DNA and the other dye specifically stains RNA of reticulocytes. A two color fluorescence measurement on the flow cytometer enables differentiation of micronucleated erythrocytes.

Grawé et al. teach flow cytometric enumeration of micronucleated polychromatic erythrocytes in mouse peripheral blood (Grawe et al. Cytometry 13, 1992, 750-758). Thiazole orange is used for discrimination between reticulocytes and normochromatic erythrocytes and Hoechst 33342 is used to detect micronucleated reticulocytes and micronucleated normochromatic erythrocytes. The peripheral blood is centrifuged to separate erythrocytes from platelets and nucleated cells. The erythrocytes are then fixed and sphered by a fixing solution containing glutaraldehyde and sodium dodecyl sulphate (SDS), and incubated at 4° C. overnight. The fixed cells are subsequently stained by dyes at 37° C.

Criswell et al. disclose a method of flow cytometric assessment of micronuclei induction using acridine orange as the discriminator of RNA and DNA (Criswell, K. A et al. Mutation Research 414, 1998, 63-75). Criswell et al. teach that the bone marrow or spleen sample is washed, and the erythrocytes are sphered and fixed in a buffer containing SDS and glutaraldehyde. The fixed cells are subsequently stained with acridine orange in staining solution chilled on ice and then incubated on ice. The stained cells are further separated from the staining solution by centrifugation prior to analysis by flow cytometry.

U.S. Pat. No. 6,100,038 (to Dertinger, et al.) discloses a flow cytometric method for analyzing micronucleated erythrocytes, particularly micronucleated reticulocytes. The method uses ultracold alcohol, from −40° C. to −90° C., to fix erythrocytes and permeate cellular membrane of erythrocytes, then simultaneously add FITC-CD71 antibody to stain reticulocytes and RNAse to digest RNA in reticulocytes, and finally add propidium iodide to stain DNA. The prepared sample is analyzed on flow cytometer by light scatter and fluorescence at two wavelengths to differentiate and enumerate micronucleated normochromatic erythrocytes and micronucleated reticulocytes of the blood sample.

U.S. Patent Application Publication No. 2006/0078949 (Offer, et al.) discloses a flow cytometry based micronucleus assay. The method uses immuno-magnetic separation to isolate and enrich CD71 young reticulocytes from whole blood sample. After applying magnetic field and washing, the enriched reticulocytes are fixed and permeated by ultra-cold methanol at −80° C. The fixed cells are subsequently mixed with RNase to degrade RNA, then stained by a nucleic acid dye.

From the above, it is apparent that all existing methods involve complicated sample preparation process, which renders these methods difficult for automation, particularly for high throughput instruments. Therefore, there exists a strong need for improved methods that simplify the sample preparation process, enable a flow cytometry measurement of micronucleated erythrocytes, without the needs of ultracold fixation, centrifugation, washing cells or low temperature incubation, and can be conveniently automated.

### SUMMARY OF THE INVENTION

The present invention is directed to a method of measurement of micronucleated erythrocyte populations. In one embodiment, the method comprises contacting a sample containing erythrocyte populations with a fluorescently labeled antibody specific to immature erythrocytes among erythrocyte populations; adding an aqueous permeation reagent to the sample and forming a permeation sample mixture; and incubating the permeation sample mixture for an incubation time sufficient to allow the permeation reagent to render cellular membrane of the erythrocyte populations permeable to RNase and nucleic acid dye; adding a RNase reagent to the permeation sample mixture to degrade RNA and to inhibit further reaction of the permeation reagent; adding a fluorescent nucleic acid dye reagent to stain DNA representing micronuclei in the erythrocyte populations and forming a final sample mixture; performing a light scatter measurement and a fluorescence measurement at two predetermined wavelengths of the final sample mixture on a flow cytometer; differentiating micronucleated erythrocyte populations from other cell types; and reporting the micronucleated erythrocyte populations of the sample.

With the method of the present invention, all sample preparation steps can be performed at room temperature, which eliminates ultracold fixation and incubation at low temperature as required by the prior art methods. Moreover, the cells are not fixed by fixatives, and no centrifugation or washing cells is required.

The advantages of the present invention will become apparent from the following description taken in conjunction with the accompanying drawings.

### BRIEF DESCRIPTION OF THE DRAWINGS

FIGS. 1A and 1B show a scattergram of forward light scatter (FS) versus side light scatter (SS) and a scattergram of FL1 versus FL4, respectively, of a normal mouse whole blood sample analyzed using the method of the present invention as described in Example 2. FS is in a linear scale and SS is in a logarithmic scale.

FIGS. 2A and 2B show a scattergram of forward light scatter (FS) versus side light scatter (SS) and a scattergram of FL1 versus FL4, respectively, of a whole blood sample of a mouse treated with methyl methane sulfonate and analyzed using the method of the present invention as described in Example 3.

### DETAILED DESCRIPTION OF THE INVENTION

The present invention provides a method of measurement of micronucleated erythrocyte populations.

The term of erythrocyte populations used herein refers to populations of mature normochromatic erythrocytes (NCE), immature erythrocytes such as reticulocytes (RET) and erythroblasts, micronucleated normochromatic erythrocytes (MNNCE), micronucleated reticulocytes, and a combination thereof from peripheral blood or bone marrow. Reticulocytes are polychromatic erythrocytes (PCE), and the micronucleated reticulocytes are also referred to as micronucleated polychromatic erythrocytes (MNPCE). The term of micronucleated erythrocyte populations refers to the erythrocyte populations containing micronuclei, i.e., micronucleated normochromatic erythrocytes, micronucleated reticulocytes, and a combination thereof.

In one embodiment, the method comprises contacting a sample containing erythrocyte populations with a fluorescently labeled antibody specific to immature erythrocytes among erythrocyte populations; adding an aqueous permeation reagent to the sample and forming a permeation sample mixture; incubating the permeation sample mixture for an incubation time sufficient to allow the permeation reagent to render cellular membrane of the erythrocyte populations permeable to RNase and nucleic acid dye; adding a RNase reagent to the permeation sample mixture to degrade RNA and to inhibit further reaction of the permeation reagent; adding a fluorescent nucleic acid dye reagent to stain DNA representing micronuclei in the erythrocyte populations, and forming a final sample mixture; performing a light scatter measurement and a fluorescence measurement at two predetermined wavelengths of the final sample mixture on a flow cytometer; differentiating micronucleated erythrocytes from other cell types; and reporting the micronucleated erythrocytes of the sample.

For the purpose of the present invention, samples are peripheral blood (also referred to as whole blood) and bone marrow from mammals, including, but not limited to humans, rodents, felines, canines, particularly rodents. More specifically, blood samples from mice and rats are used. The method of the present invention can be used in mammals (e.g., rat and man) in which the spleen is known to capture and remove circulating micronucleated erythrocytes from peripheral blood. By utilizing the cell surface markers targeted in this invention, analysis is restricted to the youngest fraction of reticulocytes which have not yet been captured by the spleen.

Blood samples should be taken from a vein that gives sufficient free flowing blood, and that can be sampled at various time points. Orbital or sublingual veinipuncture are suitable. Blood samples are rapidly added to anticoagulant containing tubes, to ensure blood clotting does not occur. Preferably, heparin is used. However, other anticoagulants, such as EDTA, sodium citrate or other commonly used anticoagulants can also be used. Blood samples are kept at room temperature, or at a temperature from about 2° C. to about 8° C., up to 4 hours, prior to the analysis of the sample using the process described above. Different from peripheral blood, which has a ratio of the erythrocyte concentration to the white blood cell concentration of about 100:1, in bone marrow, this ratio is about 2:1. Preferably, in the analysis of bone marrow samples the white blood cells can be removed from the sample first, prior to the sample being treated with the reagents used in the process of the present invention.

The antibody specific to immature erythrocytes among erythrocyte populations refers to an antibody that is specific to a surface marker of the immature erythrocytes, which is absent on mature erythrocytes. The immature erythrocytes include reticulocytes, erythroblasts, or a combination thereof. Suitable surface markers include, but are not limited to, CD71 (a transferrin receptor; Sancho et al., 1994, Biosci. Rep. 14:119-130); a 69 kDa molecule recognized by monoclonal antibody MAE15 (Tonevitsky et al., 1986, Int. J. Cancer 37:263-273); CD36 (Edelman et al. Blood, 1986, 56); a molecule referred to as Ag-Eb, antigen of erythroblasts (Levleva et al., 1976, Int. J. Cancer 17:798-805); a molecule recognized by monoclonal antibody FA6-152 (Edelman et al., 1986, Blood 67:56-63); a molecule recognized by monoclonal antibodies HAE3 and HAE9 (Levleva et al., 1986, Eksp Onkol 8:27-28); and an antigenic determinant recognized by monoclonal antibody 5F1 (Andrews et al., 1983, Blood 62:124-132); all references cited are hereby incorporated by reference in their entirety. One or more of these markers are present on mammalian species other than mice. For example, CD71 is present on human and rat reticulocytes but absent on mature erythrocytes. The antibody specific to immature erythrocytes among erythrocyte populations may bind to one or more types of leukocytes; however, since the leukocytes are substantially separated from the erythrocyte populations using the instant method, they do not affect differentiation of the erythrocyte populations.

Preferably, the antibody is a monoclonal antibody. The methods of producing a monoclonal antibody specific for the above described surface markers are known in the art, and can be used for the purpose of the present invention.

Herein, the term of fluorescently labeled antibody refers to an antibody which is conjugated or otherwise attached to a fluorescent molecule. Suitable examples of fluorescent molecules include, but are not limited to, fluorescein isothiocyanate (FITC), phycobilliproteins, Alexa dyes, and Cyanin dyes. Preferably, FITC labeled anti CD71 antibody (anti CD71-FITC antibody) is used. FITC has an excitation wavelength of about 495 nanometers (nm) and an emission wavelength about 520 nm.

In a preferred embodiment, an aliquot of a blood or bone marrow sample is diluted with an isotonic diluent, such as saline, prior to contacting the sample with the antibody specific to reticulocytes/erythroblasts. The isotonic diluent comprises one or more alkaline metal salts, such as sodium or potassium chloride. The diluent is essentially neutral and not buffered. Optionally, the diluent can further comprise one or more preservatives. Suitable examples include antimicrobials for extension of shelf life of the reagent. In one embodiment, 5-chloro-2-methyl-4-isothiazolin-3-one and 2-methyl-4-isothiazolin-3-one are used as antimicrobials. Combinations of 5-chloro-2-methyl-4-isothiazolin-3-one and 2-methyl-4-isothiazolin-3-one manufactured by Rohm and Hass, Philadelphia, Pa., are available commercially under the trade name Proclin® 150 and Proclin® 300. Other suitable preservatives such as sodium azide can also be used. Preferably, the dilution ratio is in a range from about 1:20 to about 1:40. The diluted blood sample is more convenient for mixing and further processing as described hereinafter.

After contacting with the antibody, the diluted sample is incubated for a period of time to ensure binding of the antibody to reticulocytes/erythroblasts. The incubation time varies with temperature. At room temperature, the incubation time is preferably at least 5 minutes, and at 4° C. the incubation time is preferably at least 15 minutes. For the convenience of sample preparation, preferably incubation is at room temperature, which typically includes a temperature range from about 15° C. to about 30° C. However, the incubation can be in a temperature range from about 4° C. to about 35° C. The concentration of anti CD71-FITC antibody is sufficient to saturate antigenic determinants.

Then, an aliquot of the diluted sample containing the antibody is mixed with an aqueous permeation reagent, with a dilution ratio from about 1:20 to about 1:40, forming a permeation sample mixture. The permeation sample mixture is incubated for an incubation time sufficient to allow the reaction of the permeation reagent with the cells, which renders cellular membrane of the erythrocyte populations permeable to RNase and nucleic acid dyes, and stabilizes the permeated cells as further described below. Preferably, the incubation time is from 2 minutes to 30 minutes or less, and more preferably, from about 5 minutes to about 15 minutes. For the convenience of sample preparation, preferably the incubation is at room temperature, from about 15° C. to about 30° C. However, the incubation can further be in a temperature range from about 4° C. to about 35° C.

It is noted that after the two dilutions, i.e., first by saline and second by the permeation reagent, the sample is totally diluted from about 1:400 to about 1:1600 fold. Preferably, a total dilution can be in a range from 1:400 to 1:1200. In an exemplary embodiment as shown in the Example 2, the blood sample is diluted 1:600 fold. It can be appreciated that if the blood sample is diluted in one step to such a dilution ratio, the blood sample volume is extremely small, hence, it is difficult to achieve a high accuracy in sampling. However, using the two step dilution, in either step the blood sample volume is not too small, as such the accuracy of sampling can be ensured either with manual or automated preparation.

In one embodiment, a permeation reagent as described in patent application Ser. No. 11/052,269 is used, which is also referred to as a cell permeabilization and stabilization reagent and is herein incorporated by reference in its entirety. More specifically, in one embodiment, the permeation reagent is an aqueous solution comprising N-acyl sarcosine or a salt thereof represented by the following molecular structure:

R—CO—N(CH₃)CH₂COOX

wherein R is an alkyl or alkylene group having 8 to 18 carbon atoms, and X is H, Na⁺, or K⁺, and a pH adjusting agent to adjust pH of the reagent less than 7. The permeation reagent is preferably slightly acidic, with a pH in a range from about 4 to about 6. More preferably, pH of the reagent is from about 4.6 to about 5.6, and most preferably between about 5.0 and about 5.5.

Preferably, the pH adjusting agent is a strong base or acid, therefore, a small quantity of the chemical can be used to adjust the pH within the desired range. In an exemplary embodiment, N-acyl sarcosine free acid is used, and pyrrolidine, a strong organic base, or NaOH, a strong inorganic base, is used to adjust the pH between 4 and 6. If a N-acyl sarcosine salt is used, then a strong acid, such as HCl, can be used to adjust the pH. Furthermore, an organic buffer can be used to maintain the pH. In one exemplary embodiment, succinic acid is used, which has a pKa₁ of 4.19 and pKa₂ of 5.57. The concentration of N-lauryl sarcosine is preferably from about 1.25 mM to about 2.0 mM, and more preferably from about 1.4 mM to about 1.8 mM. In one exemplary embodiment, the concentration of N-lauryl sarcosine is 1.6 mM.

The permeation reagent has a low ionic strength defined by a conductivity of less than 9.0 mS/cm, preferably less than 3.0 mS/cm, and more preferably less than 1.2 mS/cm.

It has been found that upon exposing the cells to the permeation reagent, intracellular protein aggregation is more effective under a low ionic strength. For the purpose of the present invention, the ionic strength of the aqueous reagent composition is defined by conductivity of the reagent. It is believed that intracellular protein aggregation is necessary to conserve cell integrity after permeation. When the ionic strength is too high, for example when the conductivity of the reagent is 9.0 mS/cm or higher, the reagent can no longer aggregate intracellular proteins, and the cells lose their integrity. Preferably, the permeation reagent has a conductivity less than 3.0 mS/cm, and more preferably less than 1.2 mS/cm. Since ionic compounds, such as salts, are the major contributors of the ionic strength of the reagent, preferably the reagent has a low salt concentration.

The surfactant in the concentration described above have the property of causing aggregation of proteins at a slightly acidic pH, which does not denature the intracellular and surface antigen sites, and does not destroy the cellular membrane.

Optionally, the permeation reagent can further comprise an organic osmolality adjusting agent. Suitable examples of the osmolality adjusting agent include, but are not limited to, saccharide, ethylene glycol, dimethylsulphoxide, or glycerol. Preferably, saccharide or glycerol is used. The saccharide can be a polysaccharide, such as a disaccharide, or a monosaccharide. In one exemplary embodiment as shown in Example 1, sucrose is used. Furthermore, the permeation reagent can further comprise one or more preservatives. Suitable examples include antimicrobials and antioxidants, for extension of shelf life of the reagent. The preservative can be present in an amount that does not interfere with the function of the reagent. In one embodiment, Proclin® 300 is used.

It has been found that when mixed with blood cells, the permeation reagent effectively permeates the cellular membrane which enables penetration of RNase and nucleic acid dyes into the cell; and the permeation reagent also causes intracellular protein aggregation within the cells. At the same time, however, the permeation reagent preserves the cellular constituents, such as intracellular and cell surface antigen sites, DNA and RNA molecules and cytoskeleton elements, and maintains their specific bindings with intracellular and surface markers. It has also been found that upon reaction with the permeation reagent, the erythrocytes are also sphered, which facilitates accurate measurement of erythrocytes by light scatter measurements, without the effects of cell shape and orientation.

For the purpose of the present invention, the term “cellular constituent” includes cellular components inside the cells, and on the surface of the cellular membrane such as cell surface antigen sites. While the term “intracellular constituent” refers to a cellular component inside the cells, which includes, but is not limited to, intracellular proteins, such as hemoglobin and hemoglobin variants inside the erythrocytes, cytoskeleton elements, DNA and RNA. The cytoskeleton elements include, but are not limited to, tubulin and spectrin. The term of “cellular marker” used herein includes, but is not limited to, an antibody specific to an antigen site of an intracellular protein, a cell surface antigen site, or a cytoskeleton element; a nucleic acid dye and a nucleic acid probe specific to a DNA or a RNA molecule, such as an oligonucleotide probe. Furthermore, the cellular marker specific to an intracellular constituent is also referred to as an intracellular marker. The surface marker specific to reticulocytes/erythroblasts as described above is one type of cellular markers that can be used with the instant permeation reagent.

After reaction with the permeation reagent, a RNase reagent is added to the permeation sample mixture to degrade RNA in the reticulocytes and to inhibit further reaction of permeation reagent. The RNase reagent comprises a RNAse, a buffer and one or more salts. In an exemplary embodiment, RNase A from bovine pancreas is used. Other suitable sources can also be used for the purpose of the present invention. The RNase A concentration can be from about 50 μg/ml to about 5 mg/ml, and preferably from about 100 μg/ml to about 1 mg/ml.

In addition to the function of RNase, the instant RNase reagent functions as a neutralization reagent to inhibit the further reaction of the permeation reagent. The RNase reagent is substantially isotonic and neutral. When mixed with the permeation sample mixture, the RNase reagent brings pH of the sample mixture to substantially neutral, and increases the ionic strength of the sample mixture. As such, it effectively inhibits further reaction of the permeation reagent.

Preferably, the salt in the RNase reagent is one or more alkaline metal salt, preferably alkaline metal halides, such as sodium or potassium chloride. The buffer can be an organic or inorganic buffer, which enables to raise pH of the sample mixture to above 6 and up to 8. In an exemplary embodiment, HEPES is used as the buffer. Phosphate buffered solution or other suitable buffers can also be used.

Moreover, the RNase reagent further comprises one or more preservatives. In one exemplary embodiment, Proclin® 300 is used. Other suitable preservatives can also be used. An exemplary composition of the RNase reagent is provided in Example 1.

After addition of the RNase reagent, the sample mixture is mixed and incubated at room temperature for a period of time sufficient to ensure complete degradation of RNA, preferably from about 5 minutes to about 30 minutes, and more preferably from about 10 minutes to about 20 minutes.

Subsequently, a fluorescent nucleic acid dye is added, which then forms a final sample mixture. It can be appreciated that after the degradation of RNA, only remaining nucleic acids are the DNA in the micronucleated erythrocytes. Therefore, the fluorescent nucleic acid dye used herein functions as a DNA marker. The nucleic acid specific staining identifies the micronucleated erythrocyte populations, including both micronucleated reticulocytes and micronucleated normochromatic erythrocytes.

Various nucleic acid specific dyes can be used for the purpose of the present invention, for example, propidium iodide, Hoechst dyes, Acridin Orange, Thiazol Orange, DRAQ5, 7-Actinomycin D, SYTO dye Family, TOTO dye Family, TO-PRO dye family, SYTOX dye family, DAPI, and LDS-751. In an exemplary embodiment, propidium iodide (PI) is used. Preferably, the concentration of propidium iodide in the nucleic acid dye reagent is in a range from about 0.15 mM to about 15 mM, and more preferably from about 0.75 mM to about 8 mM. The nucleic acid dye reagent further comprises bovine serum albumin for inhibiting attachment of the dye to the surface of glass vials, a preservative such as sodium azide, and a buffer to maintain pH of the nucleic acid dye reagent substantially at physiological pH. Other suitable buffer can also be used. For instance, various organic buffers, such as HEPES, can be used. An exemplary nucleic acid dye reagent composition is provided in Example 1.

The final sample mixture can be aspirated and measured on a flow cytometer equipped for light scatter and fluorescence measurements. Typically, the sample mixtures are measured on a flow cytometer in a batch by batch manner. The final sample mixture can be kept up to 4 hours at room temperature or at 4° C. prior to the measurement on the flow cytometer.

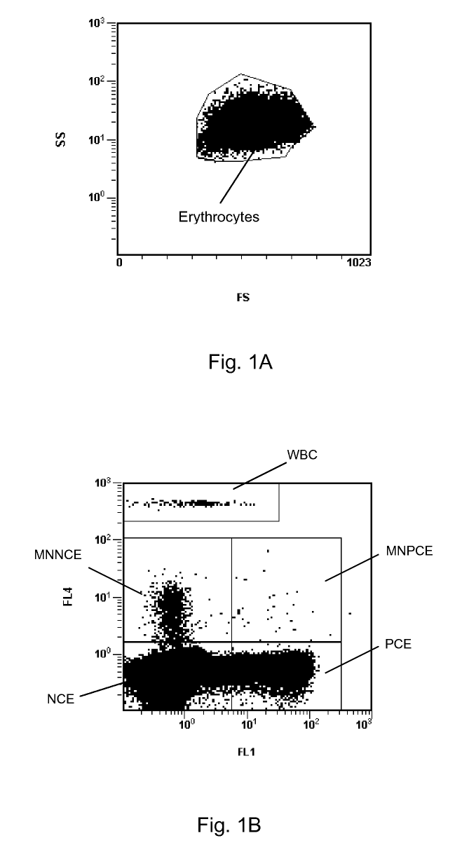

Example 2 illustrates an example using the method of the present invention for measuring micronucleated erythrocyte populations in a peripheral blood sample. A normal mouse whole blood sample was processed using the reagents described in Example 1 under the specific conditions described in Example 2. The final sample mixture was aspirated into a FC500 MCL flow cytometer, and a measurement of the forward and side scatter signals and fluorescence signals at 525 nm (FL1) and 675 nm (FL4) was made on the instrument. The obtained scattergrams of forward scatter vs. side scatter and FL1 vs. FL4 are shown in FIGS. 1A and 1B, respectively.

Herein, the term of side scatter signal, as known in flow cytometry, refers to the light scatter signal at about 90° or at the right angle from the incident light, generated by a particle or a blood cell passing through the aperture of a flow cell. The forward scatter signal refers to the light scatter signal measured less than 10° from the incident light. The term of side scatter measurement refers to the measurement of the side scatter signals by an optical detector. Most commercially available flow cytometers are equipped with a detection system which enables measurement of the forward scatter and side scatter signals.

FIG. 1A shows the gated erythrocytes on the scattergram of forward scatter (FS) vs. side scatter (SS). The erythrocytes are gated on this scattergram to remove the platelets and aggregated erythrocytes. The gated erythrocytes are displayed on the scattergram of FL1 vs. FL4 for further analysis.

In the scattergram of FL1 vs. FL4 as shown in FIG. 1B, four erythrocyte populations and white blood cells (WBC) can be identified. The lower left square encompasses FL1 negative and FL4 negative cells; these are normochromatic erythrocytes (NCE). The lower right square encompasses FL1 positive and FL4 negative cells; these are polychromatic erythrocytes (PCE) or reticulocytes. The middle left square encompasses FL1 negative and FL4 positive cells; these are micronucleated normochromatic erythrocytes (MNNCE). The middle right square encompasses FL1 positive and FL4 positive cells; these are micronucleated polychromatic erythrocytes (MNPCE) or micronucleated reticulocytes. The upper left rectangle area encompasses FL1 negative and FL4 strongly positive cells; these are leukocytes or white blood cells (WBC). If erythroblasts are present, they appear on the right hand side of white blood cells, as these cells are FL1 and FL4 strongly positive cells. It is noted that the nucleated blood cells, i.e., leukocytes and erythroblasts, are substantially separated from the erythrocyte populations, and do not affect differentiation of the erythrocyte populations.

As shown, the micronucleated erythrocyte populations, i.e., micronucleated normochromatic erythrocytes and micronucleated reticulocytes are differentiated from other cell types. The micronucleated erythrocyte populations can be reported as absolute number, percentage of the total erythrocyte populations, or percentage of a specific erythrocyte population in the blood sample. For example, the micronucleated reticulocytes can be reported as percentage of the total erythrocyte populations, or percentage in the reticulocytes of the blood sample. Alternatively, the absolute concentrations of the micronucleated erythrocyte populations in the blood sample can be reported using a reference control or a calibrator.

As shown in Example 2, the normal mouse whole blood sample had 0.16% of micronucleated reticulocytes; while in Example 3 in the blood sample of a mouse treated with methyl methane sulfonate, the micronucleated reticulocytes were 1.30%, which was substantially higher than the concentration in the normal mouse blood sample.

It can be appreciated that the method of the present invention offers various advantages over the existing methodologies. With the instant method, all sample preparation steps can be performed at room temperature, which eliminates ultracold fixation and incubation at low temperature as required by the prior art methods. Moreover, with the instant method, the cells are not fixed by fixatives, and no centrifugation or washing cells is required. Therefore, the instant method can be easily automated, which leads to high reproducibility, low labor cost, and high through put. Because of these advantages, the sample preparation procedure described in Example 2 has been successfully converted to a fully automated sample preparation process using Biomek™ NX Span 8 robot (Beckman Coulter, Inc., Fullerton, Calif.).

The following examples are illustrative of the invention and are in no way to be interpreted as limiting the scope of the invention, as defined in the claims. It will be understood that various other ingredients and proportions may be employed, in accordance with the proceeding disclosure.

### EXAMPLE 1

### Reagents Used for Preparing the Blood Sample

### Isotonic Diluent

Following isotonic diluent reagent composition was prepared:

| Component | Concentration | |
|---|---|---|
| Sodium chloride | 150 | mM |
| Proclin ® 300 | 0.5 | ml/l |

The diluent was filtered through a sterile nylon filter of 0.22 μm pore size. Proclin® 300 was obtained from Supelco, SIGMA Aldrich (St. Louis, Mo. USA).

Anti CD71-FITC antibody reagent

Following antibody reagent was prepared:

| Component | Concentration | |
|---|---|---|
| Anti CD71-FITC antibody | 25 | μg/ml |
| Bovine serum albumin | 29.2 | μM |
| Sodium chloride | 150 | mM |
| Sodium phosphate | 8 | mM |
| Sodium azide | 15.4 | mM |

pH = 7.2

Anti CD71 monoclonal antibody was obtained from Southern Biotech (Birmingham, Ala., USA). FITC was conjugated to the antibody using the method known in the art.

Permeation reagent

A permeation reagent was prepared according to the following composition:

| Component | Concentration | |
|---|---|---|
| N-lauroyl sarcosine | 1.6 | mM |
| Succinic acid | 10 | mM |
| Sucrose | 0.22 | M |
| Proclin ® 300 | 0.5 | ml/l |
| Pyrrolidine | 1.4 | ml/l |

| | |
|---|---|
| Hydrochloric acid | quantity to adjust pH at 5.2 |

More specifically, 1.4 ml of pyrrolidine were mixed with 900 ml of deionized water first. The N-lauroyl sarcosine (Fluka, SIGMA Aldrich, St. Louis, Mo. USA) was added and the mixture was agitated until N-lauroyl sarcosine was completely dissolved. Then, succinic acid, sucrose and Proclin® 300 were added and dissolved. The volume was adjusted to about 99% of the final volume of 1 liter and the pH was adjusted with hydrochloric acid to 5.2. The reagent volume was adjusted to 1 liter and the reagent was filtered through a sterile nylon filter of 0.22 μm pore size. The reagent had a conductivity of 0.9 mS/cm.

### RNase Reagent

A RNase reagent was prepared according to the following composition:

| Component | Concentration | |
|---|---|---|
| RNase A | 0.5 | mg/ml |
| HEPES | 200 | mM |
| Sodium chloride | 150 | mM |
| Proclin ® 300 | 0.5 | ml/l |

| | |
|---|---|
| Sodium hydroxide | quantity to adjust pH to 7.0 |

The reagent volume was filtered through a sterile nylon filter of 0.22 μm pore size. RNase A from bovine pancreas (SIGMA Aldrich, St Louis, USA) was used.

### Nucleic Acid Dye Reagent

A nucleic acid dye reagent was prepared according to the following composition:

| Component | Concentration | |
|---|---|---|
| Propidium iodide | 4.5 | mM |
| Bovine serum albumin | 29.2 | μM |
| Sodium phosphate | 8 | mM |
| Sodium azide | 15.4 | mM |

pH = 7.2

The reagent was filtered through a sterile nylon filter of 0.22 μm pore size. Propidium iodide was obtained from Molecular Probes (Oregon, USA).

### EXAMPLE 2

### Measurement of Micronuclei in Erythrocyte Populations in a Normal Mouse Whole Blood Sample

200 to 300 μl of a peripheral blood sample was collected from a mouse using orbital veinipuncture into a test tube containing heparin. The sample was kept at room temperature for analysis.

10 μl of the anticoagulated blood sample was diluted with 290 μl of the isotonic diluent of Example 1 in a test tube. 10 μl of anti CD71-FITC monoclonal antibody at saturating concentration was added into the diluted blood, and the test tube was vortexed and then incubated at room temperature for 30 minutes. 20 μl of the diluted sample containing anti CD71-FITC antibody was taken into a second test tube, and 400 μl of the permeation reagent of Example 1 was added and vortexed immediately. The formed permeation sample mixture was incubated at room temperature for 10 minutes. Subsequently, 400 μl of the RNase reagent of Example 1 was added into the permeation sample mixture and vortexed, then incubated at room temperature for 15 minutes. After incubation, 400 μl of the nucleic acid dye reagent of Example 1 was added and vortexed, which formed the final sample mixture. The final sample mixture was kept at room temperature prior to the analysis on the flow cytometer.

The final sample mixture was analyzed on a FC500 MCL cytometer (Beckman Coulter, Inc., Fullerton, Calif.). FIG. 1A showed the gated erythrocytes on the scattergram of forward scatter (FS) vs. side scatter (SS). FS is in a linear scale, while SS is in a logarithmic scale. The erythrocytes were gated on this scattergram to remove the platelets and aggregated erythrocytes. The gated erythrocytes were displayed on the scattergram of FL1 vs. FL4.

FIG. 1B showed the scattergram of FL1 vs. FL4 of the blood sample. Both FL1 and FL4 were in a logarithmic scale. FL1 and FL4 were the fluorescent signals of the final sample mixture detected at 525 nm and 675 nm, respectively. As shown, four erythrocyte populations and white blood cells (WBC) could be identified. The lower left square encompassed FL1 negative and FL4 negative cells; these were normochromatic erythrocytes (NCE). The lower right square encompassed FL1 positive and FL4 negative cells; these were polychromatic erythrocytes (PCE) or reticulocytes. The middle left square encompassed FL1 negative and FL4 positive cells; these were micronucleated normochromatic erythrocytes (MNNCE). The middle right square encompassed FL1 positive and FL4 positive cells; these were micronucleated polychromatic erythrocytes (MNPCE) or micronucleated reticulocytes. The upper left rectangle area encompassed FL1 negative and FL4 strongly positive cells; these were leukocytes or white blood cells (WBC).

The micronucleated erythrocyte populations, i.e., micronucleated reticulocytes and micronucleated normochromatic erythrocytes were differentiated from other cell types, by gating manually or more accurately with contour, cluster or other suitable data analysis methods. The micronucleated erythrocyte populations were reported as percentage of the total erythrocyte populations, or percentage of a specific erythrocyte population in the blood sample. In this normal mouse whole blood sample, there were 0.16% of micronucleated reticulocytes, i.e., only 0.16% of reticulocytes contained micronuclei.

### EXAMPLE 3

### Measurement of Micronuclei in Erythrocyte Populations in a Blood Sample of a Mouse Treated with a Clastogen

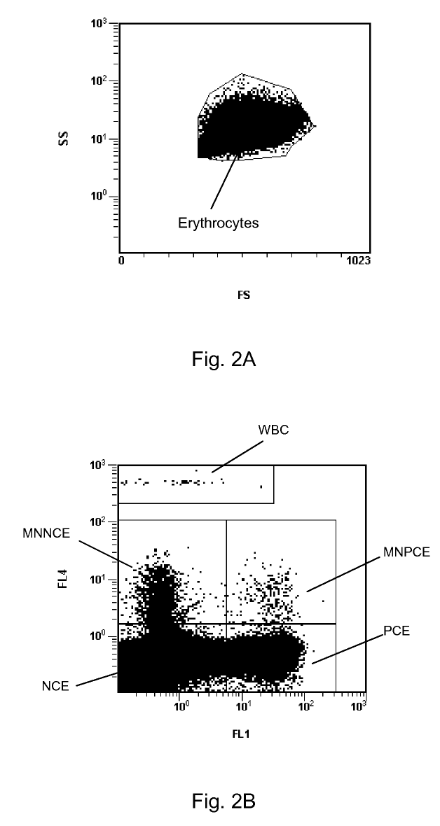

A mouse was treated with methyl methane sulfonate with a dosage of 50 mg/Kg 24 hours prior to the blood collection. The peripheral blood was collected and processed as described in Example 2, and the final sample mixture was analyzed on the same FC500 MCL cytometer with the identical instrument setting. FIGS. 2A and 2B showed the scattergrams of forward light scatter (FS) vs. side light scatter (SS) and FL1 vs. FL4, respectively.

As shown in FIG. 2B, the micronucleated reticulocytes (MNPCE) were substantially increased in the blood of the treated mouse in comparison to the normal mouse without the treatment with methyl methane sulfonate. The micronucleated reticulocytes were 1.30%, which was substantially higher than the concentration in the normal mouse blood sample.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A method of measurement of micronucleated erythrocyte populations comprising:
(a) contacting a sample containing erythrocyte populations with a fluorescently labeled antibody specific to immature erythrocytes among erythrocyte populations;
(b) adding an aqueous permeation reagent to said sample and forming a permeation sample mixture; and incubating said permeation sample mixture for an incubation time sufficient to allow said permeation reagent to render cellular membrane of said erythrocyte populations permeable to RNase and nucleic acid dye;
(c) adding a RNase reagent to said permeation sample mixture to degrade RNA and to inhibit further reaction of said permeation reagent;
(d) adding a fluorescent nucleic acid dye reagent to stain DNA representing micronuclei in said erythrocyte populations, and forming a final sample mixture;
(e) performing a light scatter measurement and a fluorescence measurement at two predetermined wavelengths of said final sample mixture on a flow cytometer;
(f) differentiating micronucleated erythrocyte populations from other cell types; and
(g) reporting said micronucleated erythrocyte populations of said sample.

2. The method of claim 1 further comprising diluting said sample by an isotonic diluent prior to the addition of said antibody.

3. The method of claim 1, wherein said immature erythrocytes comprise reticulocytes, erythroblasts, or a combination thereof.

4. The method of claim 1, wherein said micronucleated erythrocyte populations comprise micronucleated reticulocytes, micronucleated normochromatic erythrocytes, or a combination thereof.

5. The method of claim 1, wherein said reporting said micronucleated erythrocyte populations comprises reporting percentage or absolute number of micronucleated reticulocytes in said blood sample, reporting percentage or absolute number of micronucleated normochromatic erythrocytes in said blood sample, or a combination thereof.

6. The method of claim 1, wherein said permeation sample mixture is incubated at a temperature from about 4° C. to about 35° C.

7. The method of claim 1, wherein said permeation sample mixture is incubated at a temperature from about 15° C. to about 30° C.

8. The method of claim 1, wherein said incubation time is from 2 minutes to 30 minutes.

9. The method of claim 1, wherein said antibody is monoclonal anti CD71 antibody.

10. The method of claim 1, wherein said fluorescent nucleic acid dye reagent comprises propidium iodide.

11. The method of claim 1, wherein said fluorescent nucleic acid dye reagent comprises a dye selected from the group consisting of Hoechst dyes, acridin orange, thiazol orange, DRAQ5, 7-actinomycin D, SYTO dye family, TOTO dye family, TO-PRO dye family, SYTOX dye family, DAPI, and LDS-751.

12. The method of claim 1, wherein said permeation reagent comprises a surfactant and has a pH in a range from about 4 to about 6.

13. The method of claim 11, wherein said surfactant is N-acyl sarcosine or a salt thereof represented by following molecular structure:
R—CO—N(CH₃)CH₂COOX
wherein R is an alkyl or alkyene group having 8 to 18 carbon atoms, and X is H, Na⁺, or K⁺.

14. The method of claim 1, wherein said RNase reagent comprises a RNase, a buffer and one or more salts.

15. The method of claim 1, wherein said sample is a peripheral blood sample or a bone marrow sample.

16. A method of measurement of micronucleated erythrocyte populations comprising:
(a) diluting an aliquot of a sample containing erythrocyte populations with an isotonic diluent;
(b) contacting diluted sample with a fluorescently labeled antibody specific to immature erythrocytes among erythrocyte populations and forming an antibody labeled sample mixture;
(c) mixing an aliquot of said antibody labeled sample mixture with an aqueous permeation reagent and forming a permeation sample mixture; and incubating said permeation sample mixture for an incubation time sufficient to allow said permeation reagent to render cellular membrane of said erythrocyte populations permeable to RNase and nucleic acid dye;
(d) adding a RNase reagent to said permeation sample mixture to degrade RNA and to inhibit further reaction of said permeation reagent;
(e) adding a fluorescent nucleic acid dye reagent to stain DNA representing micronuclei in said erythrocyte populations, and forming a final sample mixture;
(f) performing a light scatter measurement and a fluorescence measurement at two predetermined wavelengths of said final sample mixture on a flow cytometer;
(g) differentiating micronucleated erythrocyte populations from other cell types; and
(h) reporting said micronucleated erythrocyte populations of said sample.

17. The method of claim 16, wherein said immature erythrocytes comprise reticulocytes, erythroblasts, or a combination thereof.

18. The method of claim 16, wherein said micronucleated erythrocyte populations comprise micronucleated reticulocytes, micronucleated normochromatic erythrocytes, or a combination thereof.

19. The method of claim 16, wherein said permeation sample mixture is incubated at a temperature from about 4° C. to about 35° C.

20. The method of claim 16, wherein said antibody is monoclonal anti CD71 antibody.

21. The method of claim 16, wherein said incubation time is from 2 minutes to up to 30 minutes.

22. The method of claim 16, wherein said fluorescent nucleic acid dye reagent comprises a dye selected from the group consisting of propidium iodide, Hoechst dyes, acridin orange, thiazol orange, DRAQ5, 7-actinomycin D, SYTO dye family, TOTO dye family, TO-PRO dye family, SYTOX dye family, DAPI, and LDS-751.
